(19) United States
(12) Patent Application Publication
(10) Pub. No.: US 2021/0046260 A1
(43) Pub. Date: Feb. 18, 2021

(54) AN APPARATUS AND METHOD FOR ADMINSTRING TERPENE- ENRICHED VAPOR

(71) Applicant: BUZZELET DEVELOPMENT AND TECHNOLOGIES LTD, Or-Akiva (IL)

(72) Inventors: Noa RAZ, Gizo (IL); Aharon M. EYAL, Jerusalem (IL); Carmi RAZ, Gizo (IL)

(21) Appl. No.: 16/980,396

(22) PCT Filed: Mar. 13, 2019
(86) PCT No.: PCT/IB2019/052051

§ 371 Date: Sep. 13, 2020

Related U.S. Application Data
(60) Provisional application No. 62/814,305, filed on Mar. 6, 2019.
(60) Provisional application No. 62/755,521, filed on Nov. 4, 2018.
(60) Provisional application No. 62/642,950, filed on Mar. 14, 2018.

(51) Int. Cl. A61M 11/04; A61M 15/06; A24F 40/30; A24F 40/40; A24F 40/57; A24F 40/10

(52) U.S. Cl. CPC A61M 11/042; A61M 15/06; A24F 40/10; A24F 40/40; A24F 40/57; A24F 40/30

(57) ABSTRACT

An inhalation device is described, which inhalation device comprises an inlet for vapors and/or for aerosol, at least one terpene holding element configured to hold a selected terpene composition, and an outlet for vapors and/or for aerosol and wherein the device is configured to receive vapors and/or aerosol through the inlet, move the received vapors and/or aerosol through the device, infuse terpene composition into the moved vapors and/or aerosol and generate and emit terpene-enriched vapors and/or aerosol through the outlet, wherein at least one of the inlet, the holding element and the outlet is configured to limit the concentration of terpenes in the emitted terpenes-enriched vapors and/or aerosol to less than one percentage (by weight), when the moved vapors and/or aerosol is at 10 degrees Celsius and at atmospheric pressure.

### CROSS-REFERENCE TO RELATED APPLICATIONS

The present application claims the benefit of U.S. Provisional Application Ser. No. 62/642,950 filed Mar. 14, 2018, U.S. Provisional Application Ser. No. 62/755,521 filed Nov. 4, 2018 and U.S. Provisional Application Ser. No. 62/814,305 filed Mar. 6, 2019, the disclosures of which are expressly incorporated by reference herein in their entireties.

### FIELD OF THE INVENTION

The field of art to which this invention generally pertains is an apparatus for enriching vapors and/or an aerosol with selected amounts of selected terpene compositions for therapeutic and recreational use.

### BACKGROUND OF THE INVENTION

Terpenes play an important therapeutic role on their own as well as in combination with cannabinoids due to their particular and synergistic therapeutic properties. Terpene-enriched cannabinoid compositions enhance therapeutic effects.

Enriching cannabinoid compositions with selected terpenes compositions, to enhance therapeutic effect, can be obtained by blending selected amount of selected terpenes in plant cannabis materials or extracts.

Processing cannabis plant material typically leads to terpenes loss so that most of cannabis products are deficient in terpene. That is particularly true for cannabis extracts and products thereof, such as cannabis tablets, cannabis gel capsules, cannabis patches, cannabis suppositories, etc. Most cannabis terpenes boiling points are in the range between about 150° C. and about 220° C. and they evaporate, at least partially, during cannabis buds drying, during solvent separation from extracts and during decarboxylation. Monoterpenes are lost at a rate greater than that of terpenes with a higher molecular weight and terpenes carrying no hydroxyl groups are lost at a rate greater than that of terpenes that do carry hydroxyl groups.

The invention provides an alternative approach.

### SUMMARY OF THE INVENTION

The invention comprises-an apparatus and a method for enriching therapeutic vapors and/or an aerosol with selected amounts of selected terpene compositions during an inhalation and/or spraying apparatus. Also provided is a method of using said apparatus.

The invention solves a prior art operation primary constrain, wherein heating cannabinoid and terpene composition at a single heating apparatus results in evaporating terpenes prior to cannabinoids, preventing an integration of the two components.

An inhalation device, comprising an inlet for vapors and/or for aerosol at least one terpene holding element configured to hold a selected terpene composition, liquid terpenes, and an outlet for vapors and/or for aerosol and wherein the device is configured to receive vapors and/or aerosol through the inlet, move the received vapors and/or aerosol through the device, infuse terpene composition into the moved vapors and/or aerosol and generate and emit terpene-enriched vapors and/or aerosol through the outlet, wherein at least one of the inlet, the holding element and the outlet is configured to limit the concentration of terpenes in the emitted terpenes-enriched vapors and/or aerosol to less than one percentage (by weight), when the moved vapors and/or aerosol is at 10 degrees Celsius and at atmospheric pressure.

According to an embodiment, provided is an inhalation device wherein at least one of the inlet, the holding element and the outlet is configured to adjust the concentration of terpenes in the emitted terpenes-enriched vapors and/or aerosol to at least one percent by weight, when the moved vapors and/or aerosol is at 30 degrees Celsius and at atmospheric pressure.

According to an embodiment, provided is an inhalation device wherein the terpene composition comprises at least one terpene selected from the group consisting of Hemiterpenes, Monoterpenes, Sesquiterpenes, Diterpenes, Sesterterpenes, Triterpenes, Sesquarterpenes, Tetraterpenes, Polyterpenes, Norisoprenoids and combinations thereof.

According to an embodiment, provided is an inhalation device wherein the terpene composition contains a single terpene.

According to an embodiment, provided is an inhalation device wherein the terpene composition comprises at least one terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, provided is an inhalation device wherein the terpene holding element is a perforated material and wherein the held terpene composition is dispersed in the perforated material.

According to an embodiment, provided is an inhalation device wherein the terpene holding element comprises cellulose.

According to an embodiment, provided is an inhalation device wherein the vapors and/or aerosol contact at least one surface of the terpene holding element.

According to an embodiment, provided is an inhalation device wherein at least a fraction of the moved vapors and/or aerosol passes through the terpene holding element.

According to an embodiment, provided is an inhalation device wherein the terpene holding element comprises a container, and wherein the container comprises at least one opening.

According to an embodiment, provided is an inhalation device, wherein the terpene holding element comprises an element made of fibers, and wherein the element is at least partially connected to the opening.

According to an embodiment, provided is an inhalation device, wherein the container comprises a wall and wherein the terpene holding element comprises a moveable and flexible element in at least a part of the wall.

According to an embodiment, provided is an inhalation device, wherein the flexible element is configured to release on movement a pre-determent amount of the terpene composition from the container.

According to an embodiment, provided is an inhalation device, wherein at least one of the at least one opening comprises a funnel element.

According to an embodiment, provided is an inhalation device, wherein the funnel element is configured to generate suction, when moved vapors and/or aerosol pass through the inhalation device.

According to an embodiment, provided is an inhalation device, further comprising an activation module, wherein the activation module is a button or an electronic sensor.

According to an embodiment, provided is an inhalation device, wherein the activation module is configured to release a pre-determent amount of the terpene composition from the container, when activated.

According to an embodiment, provided is an inhalation device, further comprising a spray unit configured to generate the enriched aerosol.

According to an embodiment, provided is an inhalation device, further comprising an adsorption unit configured to adsorb a selected amount of the terpenes composition from the container.

According to an embodiment, provided is an inhalation device, configured to attach to at least one of an enriched gas inhaler, a therapeutic inhalation device, an inhaler, a smoking device, a medical cannabis inhalation device, a cigarette and a cigar.

According to an embodiment, provided is an inhalation device wherein the outlet comprises at least one of a mouthpiece, an inhalation mask and an inhalation tube.

According to an embodiment, provided is an inhalation device, configured to generate the enriched vapors and/or aerosol in an amount sufficient for at least ten puffs of an adult.

According to an embodiment, provided is an inhalation device, wherein the terpene holding element is selected from the group consisting of a sheaf of fibers, a sponge, a paper, a carton, a cardboard and a cotton piece.

According to an embodiment, provided is a method for treating a patient comprising providing the inhalation device loading a selected terpene composition onto the holding element, generating the enriched vapors and/or aerosol and administering the enriched vapors and/or aerosol to the patient for inhalation.

According to an embodiment, provided is a method for providing terpene-enriched vapors and/or terpene-enriched aerosol comprising inhaling the inhalation device.

According to an embodiment, provided is an apparatus for enriching vapors and/or an aerosol with selected amount of a selected terpene composition, comprises (i) a terpene container configured to hold a selected terpene composition, which container comprises at least one container opening; (ii) a terpene-release control, configured to release a selected amount of a terpene-composition liquid and/or to release a selected amount of terpene-composition vapors from the terpene container through at least one container opening into vapors and/or aerosol in contact with the opening, generating thereby enriched vapors and/or an enriched aerosol, wherein at least one of the opening and the control is configured to limit the concentration of terpenes in the enriched vapors and/or an enriched aerosol to less than one percent by weight, when vapors and/or aerosol in contact with the opening is at 10 degrees Celsius and atmospheric pressure.

According to an embodiment, provided is an apparatus wherein at least one of the opening and the control is configured to is configured to adjust the concentration of terpenes in the enriched vapors and/or an enriched aerosol to at least one percent by weight, when vapors and/or aerosol in contact with the opening is at 30 degrees Celsius and at atmospheric pressure.

According to an embodiment, the apparatus further comprises a chamber, wherein the terpene container is attached to the chamber and wherein the at least one terpene container opening is open to the chamber.

According to an embodiment, the apparatus comprises a chamber, wherein the chamber comprises at least one chamber opening, which chamber opening is configured to provide the generated terpene-enriched vapors and/or terpene-enriched aerosol. According to an embodiment, the chamber optionally comprises an adsorption unit, configured to adsorb terpene-composition liquid, wherein the terpene container and terpene-release control are configured to release the terpene-composition liquid on to said adsorption unit.

According to an embodiment, the apparatus further comprises a holding unit configured for holding a selected therapeutic composition, wherein the therapeutic composition comprises at least one non-terpene volatile component. According to an embodiment, the therapeutic-composition holding unit comprises a therapeutic-composition container. According to an embodiment, the therapeutic-composition holding unit comprises at least two separate containers, one of which is the terpene container and the other is the therapeutic composition holding container.

According to an embodiment, the therapeutic composition comprises at least one cannabinoid.

According to an embodiment, the apparatus further comprises (i) a heating unit configured to heat the therapeutic composition; (ii) a heating control.

According to an embodiment, the heating unit in the apparatus is configured to heat the therapeutic composition to a temperature sufficient for evaporating a selected amount of the non-terpene volatile component, whereby vapors of the volatile component are formed.

According to an embodiment, the selected amount of evaporated therapeutic composition in the apparatus is at least 3 times greater than the selected amount of selected terpene-composition.

According to an embodiment, the ratio between the selected amount of evaporated therapeutic composition and the selected amount of terpene-composition is adjustable According to an embodiment, the apparatus optionally comprises a ratio-adjusting control.

According to an embodiment, the ratio-adjusting control and the terpene-release control are configured to operate in coordination.

According to an embodiment, the apparatus comprises a terpene container and a therapeutic-composition holding unit, wherein the terpene container and holding unit are configured so that at least a fraction of the selected amount of selected terpene-composition is released into the vapors of the volatile component.

According to an embodiment, the heating unit in the apparatus is configured to heat the chamber to a temperature sufficient for maintaining in vapor form at least a fraction of said non-terpene volatile component.

According to an embodiment, the apparatus comprises a terpene container, an adsorption unit and a therapeutic-composition holding unit, wherein the terpene container, the adsorption unit and the therapeutic-composition holding unit are configured so that at least a fraction of the vapors of the volatile component pass through said adsorption unit.

According to an embodiment, the apparatus comprises a heating control and a terpene-release control, configured to operate in coordination.

According to an embodiment, the chamber opening in the apparatus further comprises an inhalation unit.

According to an embodiment, the apparatus further comprises a spray unit configured to generate a selected terpene-composition comprising aerosol.

According to an embodiment, the apparatus comprises a spray unit configured to generate aerosol comprises a selected amount of the selected terpene composition and a selected amount of the selected therapeutic composition, and a spray-release control, configured to release a selected amount of the aerosol.

According to an embodiment, the spray-release control and a terpene-release control are configured to operate in coordination.

According to an embodiment, the ratio between the selected amount of the selected terpene composition and the selected amount of the therapeutic composition in the aerosol is adjustable, optionally using a ratio-adjusting control.

According to an embodiment, the adjusting control and the terpene-release control are configured to operate in coordination.

According to an embodiment, the apparatus comprises a terpene container, wherein said terpene container is replaceable.

According to an embodiment, the apparatus comprises a therapeutic-composition holding unit, wherein the holding unit is replaceable.

According to an embodiment, the apparatus comprises a terpene container and a terpene-release control, wherein said terpene-release control, is configured to release between 0.01 milligram and 100 milligram of terpene composition from said terpene container.

According to an embodiment, the apparatus comprises a terpene container, wherein the volume of the terpene container is less than 0.01 liter

According to an embodiment, the apparatus comprises a chamber, wherein the volume of the chamber is less than 1 liter.

According to an embodiment, provided is a method for treating a patient comprises providing an apparatus, wherein the apparatus comprises (i) a terpene container and (ii) a terpene-release control; and wherein said container comprises a selected terpene composition, and operating said terpene-release control, whereby terpene-enriched vapors and/or terpene-enriched aerosol is provided.

According to an embodiment, a method for treating a patient comprising providing an apparatus, wherein the apparatus comprises (i) a terpene container, (ii) a terpene-release control, (iii) a chamber, (iv) optionally an adsorption unit, (v) a therapeutic composition holding unit, (vi) a heating unit, (vii) a heating control; and wherein the terpene container comprises a selected terpene composition, wherein the therapeutic composition holding unit comprises a selected therapeutic composition comprising at least one non-terpene volatile component and operating said terpene-release control and said heating control, whereby terpene-enriched therapeutic composition vapors is provided.

According to an embodiment, a method for treating a patient is provided comprising providing an apparatus, wherein the apparatus comprises (i) a terpene container, (ii) a terpene-release control, (iii) a chamber, (iv) optionally an adsorption unit, (v) a therapeutic composition holding unit, (vi) a spray unit, (vii) a spray-release control; and wherein said container comprises a selected terpene composition, wherein said holding unit comprises a selected therapeutic composition comprising at least one non-terpene volatile component and operating said terpene-release control and said spray-release control, whereby terpene-enriched therapeutic composition aerosol is provided.

According to an embodiment, said apparatus further comprises a cavity, said cavity has an inlet, said terpene container is attached to said cavity and said at least one container opening is open to said cavity.

According to an embodiment, said cavity has an outlet and said cavity outlet is configured to provide terpene-enriched vapors and/or terpene-enriched aerosol.

According to an embodiment, said cavity inlet is configured to connect to an apparatus configured to generate a selected amount of vapors and/or aerosol of a therapeutic composition. According to an embodiment, said cavity inlet is configured to connect to a vaporizer configured to provide a selected amount of a therapeutic composition.

According to an embodiment, said cavity is configured for passing through of a selected amount of said vapors and/or aerosol of said therapeutic composition.

According to an embodiment, said terpene-release control, is configured to release a selected amount of said terpene composition, which amount is proportional to the amount of said therapeutic composition vapors and/or aerosol of a therapeutic composition. According to an embodiment, said terpene-release rate is controlled by the amount of said vapors and/or aerosol of said therapeutic composition.

According to an embodiment, said terpene-release control is configured to uses the Venturi effect.

According to an embodiment, said cavity is configured to mix said selected amount of vapors and/or aerosol of said therapeutic composition and said selected amount of terpene-composition.

According to an embodiment, said terpene-release control is configured to adjust the proportion between the selected amount of vapors and/or aerosol of said therapeutic composition and the selected amount of terpene-composition.

According to an embodiment, said cavity inlet is adjustable.

According to an embodiment, said apparatus comprises an adjustable removable coordinator.

According to an embodiment, provided is a therapeutic add-on apparatus comprising: (i) an inlet for vapors and/or aerosol; (ii) an outlet; (iii) at least one container attachment assembly including a controllable opening; (iv) at least one container adapted to hold a selected terpene composition; and (v) a mixing chamber, wherein said controllable opening is configured to transfer a selected amount of said selected terpene composition to said mixing chamber.

According to an embodiment, said mixing chamber comprises a platform for holding a selected amount of said selected terpene composition.

According to an embodiment, said controllable opening further comprises an aerosol generation unit.

According to an embodiment, said controllable opening is adjusted according to the amount of vapors and/or aerosol entered through said inlet. According to an embodiment, said controllable opening is adjustable.

According to an embodiment, said controllable opening is connected to a control unit.
According to an embodiment, said controllable opening is configured to transfer between 0.01 milligram and 100 milligrams of said selected terpene composition.

According to an embodiment, said container is replaceable. According to an embodiment, the volume of said container is less than 0.01 liter. According to an embodiment, the volume of said mixing chamber is less than 1 liter.

According to an embodiment, said inlet for vapors and/or aerosol can be adapted to connect to a vaporizer configured to provide a therapeutic composition. According to an embodiment, said apparatus comprises an additional unit comprising at least one therapeutic-composition holding unit and at least one opening, wherein said opening is adjusted to connect to said inlet for vapors and/or aerosol.

According to an embodiment, said opening is adjusted to connect to said inlet for vapors and/or aerosol via an adjustable removable coordinator.

According to an embodiment, said therapeutic-composition holding unit is configured to hold a selected therapeutic-composition.

According to an embodiment, said therapeutic composition comprises at least one cannabinoid.

According to an embodiment, said additional unit comprises a controlled heating unit. According to an embodiment, said opening of said additional unit is adjustable. According to an embodiment, said opening of said additional unit is connected to a control unit.

According to an embodiment, said heating unit is configured to heat said therapeutic composition to a temperature sufficient for evaporating a selected amount of said therapeutic composition, whereby vapors of said volatile component are formed.

According to an embodiment, said selected amount of evaporated therapeutic composition is transferred to said mixing chamber. According to an embodiment, said selected amount of evaporated therapeutic composition is mixed with said selected amount of said terpene composition. According to an embodiment, said selected amount of evaporated therapeutic composition is at least 3 times greater than said selected amount of said terpene composition transferred to the mixing chamber.

According to an embodiment, said container and said therapeutic-composition holding unit are configured so that at least a fraction of said selected amount of said selected terpene-composition is released into the vapors of said evaporated therapeutic composition. According to an embodiment, said therapeutic-composition holding unit is replaceable.

According to an embodiment, said selected amount of evaporated therapeutic composition and said selected amount of said terpene-composition transferred to said mixing chamber are adjustable.

According to an embodiment, said vapors and/or aerosol of said therapeutic composition are configured to release said terpene composition from said container. According to an embodiment, said release is configured to use the Venturi effect.

According to an embodiment, provided is a method for treating a patient comprising providing said apparatus, wherein said container comprises a selected terpene composition, and operating said terpene-release control, whereby terpene-enriched vapors and/or terpene-enriched aerosol is provided.

According to an embodiment, provided is a method for treating a patient comprising providing said apparatus, wherein said container comprises a selected terpene composition, and operating said terpene-release control, whereby terpene-enriched vapors and/or terpene-enriched aerosol is provided.

According to an embodiment, provided is a method for treating a patient comprising providing said apparatus, wherein said at least one container comprises a selected terpene composition, wherein additional unit container comprises a therapeutic composition comprising at least one non-terpene volatile component and operating said terpene-release control and said heating control, whereby terpene-enriched therapeutic composition vapors is provided.

### BRIEF DESCRIPTION OF THE FIGURES

FIG. 1 shows an inhalation device, in accordance with exemplary embodiment of present invention.

FIG. 2 shows an inhalation device, in accordance with exemplary embodiment of present invention.

FIG. 3 shows exemplary uses of an inhalation device, in accordance with exemplary embodiment of present invention.

FIG. 4 shows an apparatus, in accordance with exemplary embodiment of present invention.

FIG. 5 shows an apparatus, in accordance with exemplary embodiment of present invention.

### DETAILED DESCRIPTION OF THE INVENTION

### Definitions

As used herein the term “cavity” refers to an empty space within a solid object.

As used herein the terms “cavity” and “chamber” are used interchangeably.

As used herein the terms “therapeutic composition” and “non-terpene volatile component” refer to a therapeutic composition comprising at least one non-terpene volatile component.

As used herein the term “aerosol” refers to a stream of small droplets or a mist of liquid particles.

As used herein the term “heating” refers to direct or indirect heating. Any heating unit is suitable.

As used herein the terms “evaporating” and “evaporated” refer to conversion of a solid or a liquid compound into a vapor form, e.g. as in boiling or sublimation.

As used herein the term “adsorption” is defined to include any form of wetting, e.g. wetting by a liquid that stays on the surface.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description, which follows, and in part will be obvious from the description, or may be learned by practice of the invention. It is to be understood that both the foregoing general description and the following detailed description are exemplary and explanatory only and are not restrictive of the invention, as claimed.

An inhalation device, comprising an inlet for vapors and/or for aerosol at least one terpene holding element configured to hold a selected terpene composition, liquid terpenes, and an outlet for vapors and/or for aerosol and wherein the device is configured to receive vapors and/or aerosol through the inlet, move the received vapors and/or aerosol through the device, infuse terpene composition into the moved vapors and/or aerosol and generate and emit terpene-enriched vapors and/or aerosol through the outlet, wherein at least one of the inlet, the holding element and the outlet is configured to limit the concentration of terpenes in the emitted terpenes-enriched vapors and/or aerosol to less than one percentage (by weight), when the moved vapors and/or aerosol is at 10 degrees Celsius and at atmospheric pressure.

According to an embodiment, provided is an inhalation device wherein at least one of the inlet, the holding element and the outlet is configured to adjust the concentration of terpenes in the emitted terpenes-enriched vapors and/or aerosol to at least one percent by weight, when the moved vapors and/or aerosol is at 30 degrees Celsius and at atmospheric pressure.

According to an embodiment, provided is an inhalation device wherein the terpene composition comprises at least one terpene selected from the group consisting of Hemiterpenes, Monoterpenes, Sesquiterpenes, Diterpenes, Sesterterpenes, Triterpenes, Sesquarterpenes, Tetraterpenes, Polyterpenes, Norisoprenoids and combinations thereof.

According to an embodiment, provided is an inhalation device wherein the terpene composition contains a single terpene.

According to an embodiment, provided is an inhalation device wherein the terpene composition comprises at least one terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, amyrin, thujone, citronellol, pulegone, cycloartenol, cymene, sabinene, carene, terpinene, fenchol, isopulegol, guaiol, phellandrene, eudesmol, ocimene, cardinol, elemene, friedelin, carvacrol, eugenol, camphor, menthol, iso-menthone, neral, gerial, viridiflorol, germacrene, thymol, Menth-2-en-1-ol, farensol, carotol, myrtenol and combinations thereof.

According to an embodiment, provided is an inhalation device wherein the terpene holding element is a perforated material and wherein the held terpene composition is dispersed in the perforated material.

According to an embodiment, provided is an inhalation device wherein the terpene holding element comprises cellulose.

According to an embodiment, provided is an inhalation device wherein the vapors and/or aerosol contact at least one surface of the terpene holding element.

According to an embodiment, provided is an inhalation device wherein at least a fraction of the moved vapors and/or aerosol passes through the terpene holding element.

According to an embodiment, provided is an inhalation device wherein the terpene holding element comprises a container, and wherein the container comprises at least one opening.

According to an embodiment, provided is an inhalation device, wherein the terpene holding element comprises an element made of fibers, and wherein the element is at least partially connected to the opening.

According to an embodiment, provided is an inhalation device, wherein the container comprises a wall and wherein the terpene holding element comprises a moveable and flexible element in at least a part of the wall.

According to an embodiment, provided is an inhalation device, wherein the flexible element is configured to release on movement a pre-determent amount of the terpene composition from the container.

According to an embodiment, provided is an inhalation device, wherein at least one of the at least one opening comprises a funnel element.

According to an embodiment, provided is an inhalation device, wherein the funnel element is configured to generate suction, when moved vapors and/or aerosol pass through the inhalation device.

According to an embodiment, provided is an inhalation device, further comprising an activation module, wherein the activation module is a button or an electronic sensor.

According to an embodiment, provided is an inhalation device, wherein the activation module is configured to release a pre-determent amount of the terpene composition from the container, when activated.

According to an embodiment, provided is an inhalation device, further comprising a spray unit configured to generate the enriched aerosol.

According to an embodiment, provided is an inhalation device, further comprising an adsorption unit configured to adsorb a selected amount of the terpenes composition from the container.

According to an embodiment, provided is an inhalation device, configured to attach to at least one of an enriched gas inhaler, a therapeutic inhalation device, an inhaler, a smoking device, a medical cannabis inhalation device, a cigarette and a cigar.

According to an embodiment, provided is an inhalation device wherein the outlet comprises at least one of a mouthpiece, an inhalation mask and an inhalation tube.

According to an embodiment, provided is an inhalation device, configured to generate the enriched vapors and/or aerosol in an amount sufficient for at least ten puffs of an adult.

According to an embodiment, provided is an inhalation device, wherein the terpene holding element is selected from the group consisting of a sheaf of fibers, a sponge, a paper, a carton, a cardboard and a cotton piece.

According to an embodiment, provided is a method for treating a patient comprising providing the inhalation device loading a selected terpene composition onto the holding element, generating the enriched vapors and/or aerosol and administering the enriched vapors and/or aerosol to the patient for inhalation.

According to an embodiment, provided is a method for providing terpene-enriched vapors and/or terpene-enriched aerosol comprising inhaling the inhalation device.

According to an embodiment, provided is an apparatus for enriching vapors and/or an aerosol with selected amount of a selected terpene composition, comprises (i) a terpene container configured to hold a selected terpene composition, which container comprises at least one container opening; (ii) a terpene-release control, configured to release a selected amount of a terpene-composition liquid and/or to release a selected amount of terpene-composition vapors from the terpene container through at least one container opening into vapors and/or aerosol in contact with the opening, generating thereby enriched vapors and/or an enriched aerosol, wherein at least one of the opening and the control is configured to limit the concentration of terpenes in the enriched vapors and/or an enriched aerosol to less than one percent by weight, when vapors and/or aerosol in contact with the opening is at 10 degrees Celsius and atmospheric pressure.

According to an embodiment, provided is an apparatus wherein at least one of the opening and the control is configured to is configured to adjust the concentration of terpenes in the enriched vapors and/or an enriched aerosol to at least one percent by weight, when vapors and/or aerosol in contact with the opening is at 30 degrees Celsius and at atmospheric pressure.

According to an embodiment, a terpene container refers to any container and/or a receptacle or enclosure unit of holding a product used in storage, including a cartridge, a tube, a can, etc. According to an embodiment, the terpene container is sealed at all non-opening surfaces, configured to prevent uncontrolled terpene evaporation; According to an embodiment, selected terpene composition refers to any selected terpene composition, including compositions according to U.S. provisional patent application Ser. No. 62/639,233 filed on Mar. 6, 2018, fully incorporated here by reference. According to an embodiment, the terpene-release control comprises any controlling device, for example via using an electric, mechanical, remote control, etc.

According to an embodiment, the apparatus further comprises a chamber, wherein the terpene container is attached to the chamber and wherein the at least one terpene container opening is open to the chamber.

According to an embodiment, a chamber refers any unit for holding and/or attaching the internal containers, including a commercial vaporizer.

According to an embodiment, the apparatus comprises a chamber, wherein the chamber comprises at least one opening, which chamber opening is configured to provide the generated terpene-enriched vapors and/or terpene-enriched aerosol. According to an embodiment, the chamber optionally comprises an adsorption unit, configured to adsorb terpene-composition liquid, wherein the terpene container and terpene-release control are configured to release the terpene-composition liquid on to said adsorption unit. According to an embodiment, an adsorption unit includes any unit to adsorb the terpene-composition liquid.

According to an embodiment, the apparatus further comprises a holding unit configured for holding a selected therapeutic composition, wherein the therapeutic composition comprises at least one non-terpene volatile component. According to an embodiment, the therapeutic-composition holding unit comprises a therapeutic-composition container. According to an embodiment, the therapeutic-composition holding unit comprises at least two separate containers, one of which is the terpene container and the other is the therapeutic composition holding container. According to an embodiment, the therapeutic composition holding unit comprises a cartridge, a tube or any other form of container. According to an embodiment, the unit is sealed at all non-opening surfaces.

According to an embodiment, the said therapeutic composition comprises at least one cannabinoid. According to an embodiment, the said therapeutic composition comprises at least two cannabinoids, at least three, at least four, or at least five cannabinoids. According to an embodiment, the said therapeutic composition comprises a solution of a cannabinoid in a solvent, a cannabis plant material, a cannabis extract and a solution of said extract in a solvent. According to an embodiment, the therapeutic composition may also contain terpene composition.

According to an embodiment, the apparatus further comprises (i) a heating unit configured to heat the therapeutic composition; (ii) a heating control. According to an embodiment, the heating control comprises any controlling device, for example via using an electric, mechanical, remote control, etc.

According to an embodiment, the heating unit in the apparatus is configured to heat the therapeutic composition to a temperature sufficient for evaporating a selected amount of the non-terpene volatile component, whereby vapors of the volatile component are formed. According to an embodiment, the heating unit in the apparatus is configured to heat the therapeutic composition to a temperature sufficient for evaporating at 20%, at least 30%, at least 40%, at least 50%, at least 60%, at least 70%, or at least 80% of the non-terpene volatile component, whereby vapors of the volatile component are formed.

According to an embodiment, the selected amount of evaporated therapeutic composition in the apparatus is at least 3 times greater than the selected amount of selected terpene-composition. According to an embodiment, the amount of evaporated therapeutic composition in the apparatus is at least twice greater, at least 4 times greater, at least 5 time greater, at least 6 times greater than the selected amount of terpene-composition.

According to an embodiment, the ratio between the selected amount of evaporated therapeutic composition and the selected amount of terpene-composition is adjustable. According to an embodiment, the apparatus optionally comprises a ratio-adjusting control. According to an embodiment, the ratio-adjusting control comprises any controlling device, for example via using an electric, mechanical, remote control, etc.

According to an embodiment, the ratio-adjusting control and the terpene-release control are configured to operate in coordination.

According to an embodiment, the apparatus comprises a terpene container and a therapeutic-composition holding unit, wherein the terpene container and holding unit are configured so that at least a fraction of the selected amount of selected terpene-composition is released into the vapors of the volatile component.

According to an embodiment, the heating unit in the apparatus is configured to heat the chamber to a temperature sufficient for maintaining in vapor form at least a fraction of said non-terpene volatile component.

According to an embodiment, the apparatus comprises a terpene container, an adsorption unit and a therapeutic-composition holding unit, wherein the terpene container, the adsorption unit and the therapeutic-composition holding unit are configured so that at least a fraction of the vapors of the volatile component pass through said adsorption unit.

According to an embodiment, the apparatus comprises a heating control and a terpene-release control, configured to operate in coordination.

According to an embodiment, the chamber opening in the apparatus further comprises an inhalation unit. According to an embodiment, an inhalation unit comprises any device for delivering the evaporated therapeutic composition thorough the lungs, via nasal or mouth breathing.

According to an embodiment, the apparatus further comprises a spray unit configured to generate a selected terpene-composition comprising aerosol. According to an embodiment, a spray unit comprises a unit of conversion of a solid or a liquid compound into particles small enough to be carried in a carrier gas, e.g. air, e.g. as in an aerosol.

According to an embodiment, the apparatus comprises a spray unit configured to generate aerosol comprising a selected amount of the selected terpene composition and a selected amount of the selected therapeutic composition, and a spray-release control, configured to release a selected amount of the aerosol. According to an embodiment, the spray-release control comprises any controlling device, for example via using electric, mechanical, remote control, etc.

According to an embodiment, the spray-release control and a terpene-release control are configured to operate in coordination.

According to an embodiment, the ratio between the selected amount of the selected terpene composition and the selected amount of the therapeutic composition in the aerosol is adjustable, optionally using a ratio-adjusting control.

According to an embodiment, the ration-adjusting control and the terpene-release control are configured to operate in coordination.

According to an embodiment, the apparatus comprises a terpene container, wherein said terpene container is replaceable.

According to an embodiment, the apparatus comprises a therapeutic-composition holding unit, wherein the holding unit is replaceable.

According to an embodiment, the apparatus comprises a terpene container and a terpene-release control, wherein said terpene-release control, is configured to release between 0.01 milligram and 100 milligram of terpene composition from said terpene container. According to an embodiment, the terpene-release control is configured to release at least 0.01 milligram, at least 0.05 milligram, at least 0.1 milligram or at least 0.5 milligram of terpene composition from said terpene container. According to an embodiment, the terpene-release control is configured to release less than 100 milligram, less than 50 milligram, less than 20 milligram, or less than 10 milligram of terpene composition from said terpene container.

According to an embodiment, the apparatus comprises a terpene container, wherein the volume of the terpene container is less than 0.01 liter. According to an embodiment, the volume of the container is less than 0.02 liter, less than 0.03 liter, less than 0.05 liter.

According to an embodiment, the apparatus comprises a chamber, wherein the volume of the chamber is less than 1 liter. According to an embodiment, the apparatus comprises a chamber, wherein the volume of the chamber is less than 2 liters, less than 3 liters, less than 4 liters.

According to an embodiment, provided is a method for treating a patient comprises providing an apparatus, wherein the apparatus comprises (i) a terpene container and (ii) a terpene-release control; and wherein said container comprises a selected terpene composition, and operating said terpene-release control, whereby terpene-enriched vapors and/or terpene-enriched aerosol is provided. According to an embodiment, treating a patient is according to U.S. provisional patent application Ser. No. 62/639,233. According to an embodiment, a patient can exchange terpene container during a single treatment session to combine treatments. For example, combining compositions for treating pain and sleep disorders; combining compositions for treating anxiety and irritability, etc.

According to an embodiment, a method for treating a patient comprising providing an apparatus, wherein the apparatus comprises (i) a terpene container, (ii) a terpene-release control, (iii) a chamber, (iv) optionally an adsorption unit, (v) a holding unit, (vi) a heating unit, (vii) a heating control; (viii) optionally an adjusting control; and wherein the terpene container comprises a selected terpene composition, wherein the holding unit comprises a therapeutic composition comprising at least one non-terpene volatile component and operating said terpene-release control and said heating control, whereby terpene-enriched therapeutic composition vapors is provided. According to an embodiment, treating a patient is according to U.S. provisional patent application Ser. No. 62/639,233.

According to an embodiment, a method for treating a patient comprising providing an apparatus, wherein the apparatus comprises (i) a terpene container, (ii) a terpene-release control, (iii) a chamber, (iv) optionally an adsorption unit, (v) a holding unit, (vi) a spray unit, (vii) a spray-release control, (viii) optionally an adjusting control; and wherein said container comprises a selected terpene composition, wherein said holding unit comprises a therapeutic composition comprising at least one non-terpene volatile component and operating said terpene-release control and said spray-release control, whereby terpene-enriched therapeutic composition aerosol is provided. According to an embodiment, treating a patient is according to U.S. provisional patent application Ser. No. 62/639,233.

According to an embodiment, said apparatus further comprises a cavity, said cavity has an inlet, said terpene container is attached to said cavity and said at least one container opening is open to said cavity.

According to an embodiment, said cavity has an outlet and said cavity outlet is configured to provide terpene-enriched vapors and/or terpene-enriched aerosol.

According to an embodiment, said cavity inlet is configured to connect to an apparatus configured to generate a selected amount of vapors and/or aerosol of a therapeutic composition. According to an embodiment, said cavity inlet is configured to connect to a vaporizer configured to provide a selected amount of a therapeutic composition.

According to an embodiment, said cavity is configured for passing through of a selected amount of said vapors and/or aerosol of said therapeutic composition.

According to an embodiment, said terpene-release control, is configured to release a selected amount of said terpene composition, which amount is proportional to the amount of said therapeutic composition vapors and/or aerosol of a therapeutic composition. According to an embodiment, said terpene-release rate is controlled by the amount of said vapors and/or aerosol of said therapeutic composition.

According to an embodiment, said terpene-release control is configured to uses the Venturi effect.

According to an embodiment, said cavity is configured to mix said selected amount of vapors and/or aerosol of said therapeutic composition and said selected amount of terpene-composition.

According to an embodiment, said terpene-release control is configured to adjust the proportion between the selected amount of vapors and/or aerosol of said therapeutic composition and the selected amount of terpene-composition.

According to an embodiment, said cavity inlet is adjustable.

According to an embodiment, said apparatus comprises an adjustable removable coordinator.

According to an embodiment, provided is a therapeutic add-on apparatus comprising: (i) an inlet for vapors and/or aerosol; (ii) an outlet; (iii) at least one container attachment assembly including a controllable opening; (iv) at least one container adapted to hold a selected terpene composition; and (v) a mixing chamber, wherein said controllable opening is configured to transfer a selected amount of said selected terpene composition to said mixing chamber.

According to an embodiment, said mixing chamber comprises a platform for holding a selected amount of said selected terpene composition.

According to an embodiment, said controllable opening further comprises an aerosol generation unit.

According to an embodiment, said controllable opening is adjusted according to the amount of vapors and/or aerosol entered through said inlet. According to an embodiment, said controllable opening is adjustable.

According to an embodiment, said controllable opening is connected to a control unit.
According to an embodiment, said controllable opening is configured to transfer between 0.01 milligram and 100 milligrams of said selected terpene composition.

According to an embodiment, said container is replaceable. According to an embodiment, the volume of said container is less than 0.01 liter. According to an embodiment, the volume of said mixing chamber is less than 1 liter.

According to an embodiment, said inlet for vapors and/or aerosol can be adapted to connect to a vaporizer configured to provide a therapeutic composition. According to an embodiment, said apparatus comprises an additional unit comprising at least one therapeutic-composition holding unit and at least one opening, wherein said opening is adjusted to connect to said inlet for vapors and/or aerosol.

According to an embodiment, said opening is adjusted to connect to said inlet for vapors and/or aerosol via an adjustable removable coordinator.

According to an embodiment, said therapeutic-composition holding unit is configured to hold a selected therapeutic-composition.

According to an embodiment, said therapeutic composition comprises at least one cannabinoid.

According to an embodiment, said additional unit comprises a controlled heating unit. According to an embodiment, said opening of said additional unit is adjustable. According to an embodiment, said opening of said additional unit is connected to a control unit.

According to an embodiment, said heating unit is configured to heat said therapeutic composition to a temperature sufficient for evaporating a selected amount of said therapeutic composition, whereby vapors of said volatile component are formed.

According to an embodiment, said selected amount of evaporated therapeutic composition is transferred to said mixing chamber. According to an embodiment, said selected amount of evaporated therapeutic composition is mixed with said selected amount of said terpene composition. According to an embodiment, said selected amount of evaporated therapeutic composition is at least 3 times greater than said selected amount of said terpene composition transferred to the mixing chamber.

According to an embodiment, said container and said therapeutic-composition holding unit are configured so that at least a fraction of said selected amount of said selected terpene-composition is released into the vapors of said evaporated therapeutic composition. According to an embodiment, said therapeutic-composition holding unit is replaceable.

According to an embodiment, said selected amount of evaporated therapeutic composition and said selected amount of said terpene-composition transferred to said mixing chamber are adjustable.

According to an embodiment, said vapors and/or aerosol of said therapeutic composition are configured to release said terpene composition from said container. According to an embodiment, said release is configured to use the Venturi effect.

According to an embodiment, provided is a method for treating a patient comprising providing said apparatus, wherein said container comprises a selected terpene composition, and operating said terpene-release control, whereby terpene-enriched vapors and/or terpene-enriched aerosol is provided.

According to an embodiment, provided is a method for treating a patient comprising providing said apparatus, wherein said container comprises a selected terpene composition, and operating said terpene-release control, whereby terpene-enriched vapors and/or terpene-enriched aerosol is provided.

According to an embodiment, provided is a method for treating a patient comprising providing said apparatus, wherein said at least one container comprises a selected terpene composition, wherein an additional unit container comprises a therapeutic composition comprising at least one non-terpene volatile component and operating said terpene-release control and said heating control, whereby terpene-enriched therapeutic composition vapors is provided.

### Examples

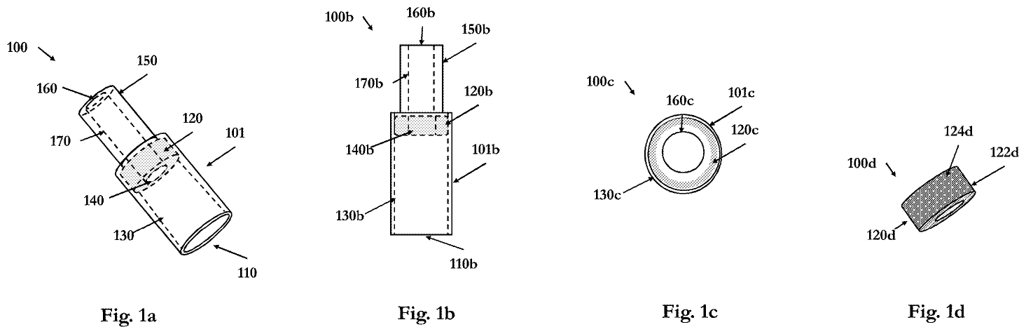

An exemplary inhalation device (100) is shown in FIG. 1a. FIG. 1b. shows a side view (100b) of the exemplary inhalation device (100) shown in FIG. 1a. FIG. 1c. shows a top down view (100c) of the exemplary inhalation device (100) shown in FIG. 1a. FIG. 1d. shows an exemplary terpene holding element that can be part of exemplary inhalation device (100)

In FIG. 1a. exemplary inhalation device (100) made from tubular body (101) with an opening (110), the inhalation device inlet. The tubular body (101) has an internal surface (130) shown here as dotted line. The inhalation device (100) comprises a terpene holding element (120) configured to hold selected terpene composition, the terpene holding element can comprise an opening (140). The inhalation device (100) comprises a mouthpiece 150 with an interior tubular cavity (170) and an opening (160) the outlet. Inhalation through outlet (160) cause vapors to enter the inhalation device from inlet (110) pass through or by terpene holding element (120) carrying liquid terpene vapor from the terpene holding element (120) through interior tubular cavity (170) via outlet (160).

FIG. 1b. shows a side view (mob) of the exemplary inhalation device (100) shown in FIG. 1a. in FIG. 1b. exemplary inhalation device (mob) made from tubular body (101b) with an opening (nob) the inhalation device inlet. The tubular body (101b) has an internal surface (130b) shown here as dotted line. a terpene holding element (120b) configured to hold selected terpene composition, the terpene holding element can comprise an opening (mob). a mouthpiece (150b) with an interior tubular cavity (170b) and an opening (160b) the outlet.

FIG. 1c. shows a top down view (100c) of the exemplary inhalation device (100) shown in FIG. 1a. in FIG. 1c. exemplary inhalation device (100c) made from tubular body (101c) with an internal surface (130c). a terpene holding element (120c) configured to hold selected terpene composition, the terpene holding element can comprise an opening, a mouthpiece with an opening (160c) the outlet.

FIG. 1d. shows (mod) exemplary terpene holding element (120d) that can be part of exemplary inhalation device (100). The exemplary terpene holding element (120d) is made from fibrous material (122d) that liquid terpene (124d) can be soaked in the fibrous material (122d).

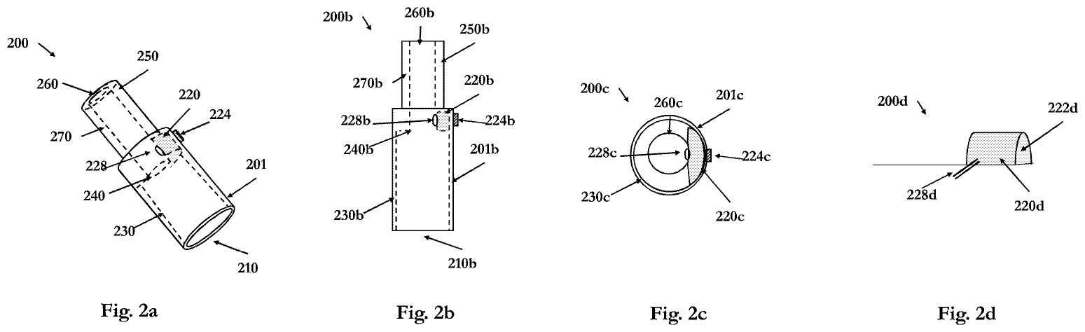

An additional exemplary inhalation device (200) is shown in FIG. 2a. FIG. 2b. shows a side view (200b) of the exemplary inhalation device (200) shown in FIG. 2a. FIG. 2c. shows a top down view (200c) of the exemplary inhalation device (200) shown in FIG. 2a. FIG. 2d. shows exemplary terpene container that can be part of exemplary inhalation device (200).

In FIG. 2a. exemplary inhalation device (200) made from tubular body (201) with an opening (210) the inhalation device inlet. The tubular body (201) has an internal surface (230) shown here as dotted line. The inhalation device (200) comprises a terpene container (220) configured to hold selected terpene composition, the terpene holding container comprise an opening (228). And can comprise an activation button (224) The inhalation device (200) comprises a mouthpiece (250) with an interior tubular cavity (270) with an opening (240) and an opening (260) the outlet. Inhalation through outlet (260) cause vapors to enter the inhalation device from inlet (210) pass next to terpene holding container opening (228) carrying liquid terpene vapor through interior tubular cavity (270) via outlet (260). In some embodiment of exemplary inhalation device (200) a pressing on button (224) will cause a selected amount of terpene to release from the terpene container (220) through terpene holding container opening (228).

FIG. 2b. shows a side view (200b) of the exemplary inhalation device (200) shown in FIG. 2. in FIG. 2b. exemplary inhalation device (200b) made from tubular body (201b) with an opening (210b) the inhalation device inlet. The tubular body (201b) has an internal surface (230b) shown here as dotted line. a terpene container (220b) configured to hold selected terpene composition, and a terpene holding container opening (228b) and can comprise an activation button (224b). The inhalation device (200b) comprises a mouthpiece (250b) with an interior tubular cavity (270b) with an opening (240b) and an opening (260b) outlet.

FIG. 2c. shows a top down view (200c) of the exemplary inhalation device (200) shown in FIG. 2a. in FIG. 2c. exemplary inhalation device (200c) made from tubular body (201c) with an internal surface (230c) a terpene container (220c) configured to hold selected terpene composition, and a terpene holding container opening (228c) and can comprise an activation button (224c). mouthpiece with an opening (260c) outlet.

FIG. 2d. shows (200d) exemplary terpene holding container (220d) that can be part of exemplary inhalation device (200). The exemplary terpene holding container (220d) has a flexible wall (222d) that can move when pressure is operated, reducing the volume of the exemplary terpene holding container (220d) and can cause liquid terpene that can be held in the terpene holding container (220d) to exit from the terpene holding container opening (228d), in some exemplary embodiment the terpene holding container opening (228d) is a funnel or a tube.

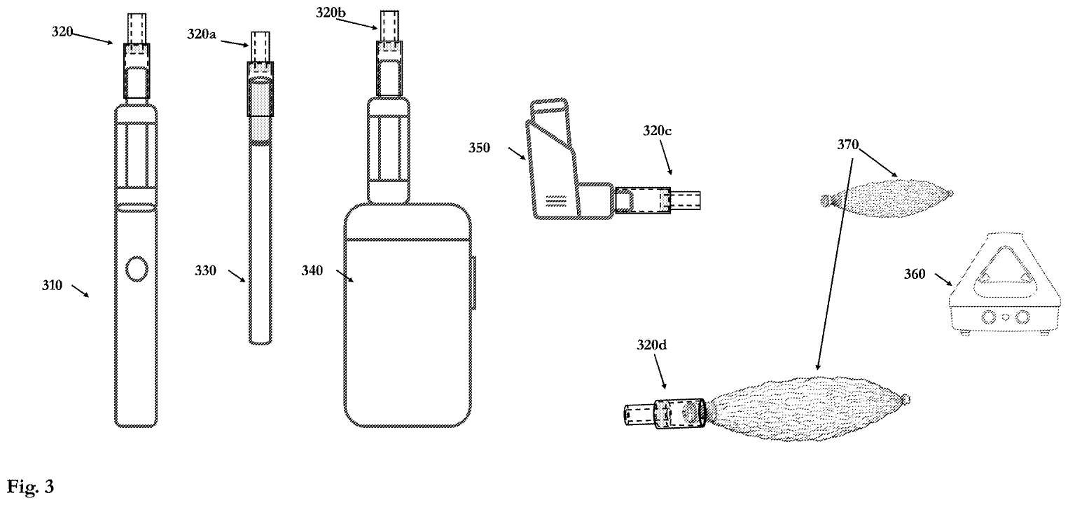

In FIG. 3. uses of an exemplary inhalation device as shown in FIG. 1 are shown, in accordance with exemplary embodiment of the present invention. 320, 320a, 320b, 320c and 320d are inhalation devices as shown in figure one. 310, 330, 340, 350, 360 are examples of existing inhalation devices and are respectively electronic pen vaporizer, cigarette, electronic vaporizer, inhaler and a desktop vaporizer. Exemplary inhalation device (320) is attached to the mouthpiece of electronic pen vaporizer (310), inhalation via the mouthpiece of inhalation device (320) and will draw vapors through electronic pen vaporizer (310). Exemplary inhalation device (320a) is attached to the mouthpiece cigarette (330), inhalation via the mouthpiece of inhalation device (320a) and will draw vapors through cigarette (330). Exemplary inhalation device (320b) is attached to the mouthpiece of electronic vaporizer (340), inhalation via the mouthpiece of inhalation device (320b) and will draw vapors through electronic vaporizer (340). Exemplary inhalation device (320c) is attached to the mouthpiece inhaler (350), inhalation via the mouthpiece of inhalation device (320c) and will draw vapors through inhaler (350). Exemplary inhalation device (320d) is attached to the mouthpiece of the desktop evaporation balloon (370), inhalation via the mouthpiece of inhalation device (320d) and will draw vapors through the desktop evaporation balloon (370).

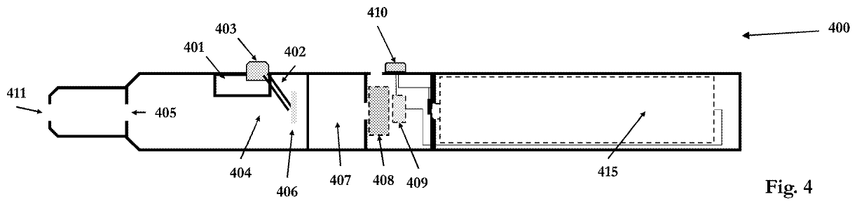

An exemplary embodiment (400) is shown in FIG. 4. It comprises a terpene container (401) configured to hold selected terpene composition, which container comprises at least one opening (402) and a terpene-release control (403), configured to release a selected amount of terpene-composition liquid and/or to release a selected amount of terpene-composition vapors from the terpene container (401) through said container opening (402). The exemplary apparatus further comprises a chamber (404), and said container is attached to said chamber so that said opening (402) is open to said chamber. In the exemplary apparatus the chamber (404) comprises at least one opening (405), which chamber opening is configured to provide terpene-enriched vapors. The exemplary apparatus further comprises an adsorption unit (406), configured to adsorb terpene-composition liquid, wherein said terpene container (401) and terpene-release control (403) are configured to release the terpene-composition liquid on to the adsorption unit (406).

The exemplary apparatus further comprises a unit and/or container (407), configured for holding a therapeutic composition comprises at least one non-terpene volatile component. The exemplary apparatus further comprises a heating unit (408), configured to heat said therapeutic composition to a temperature sufficient to evaporate and to maintain in vapor form at least a fraction of the non-terpene volatile component. The exemplary apparatus, wherein said terpene container (401) and said holding unit (407) are configured so that at least a fraction of the selected amount of terpene-composition is released into the vapors of said volatile component. The exemplary apparatus, wherein said terpene container (401), said adsorption unit (406) and said holding unit (407) are configured so that at least a fraction of the vapors of said volatile component pass through the adsorption unit (406). The exemplary apparatus further comprises a heating control (409) and power source (415), wherein said heating control (409) and terpene-release control (403) are configured to operate in coordination. The exemplary apparatus further comprises a ratio-adjusting control (410), configured to adjust the ratio between the amount of evaporated therapeutic composition and the amount of terpene-composition, wherein said ratio-adjusting control (410) and terpene-release control (403) are configured to operate in coordination. The exemplary apparatus further comprises an inhalation unit (411).

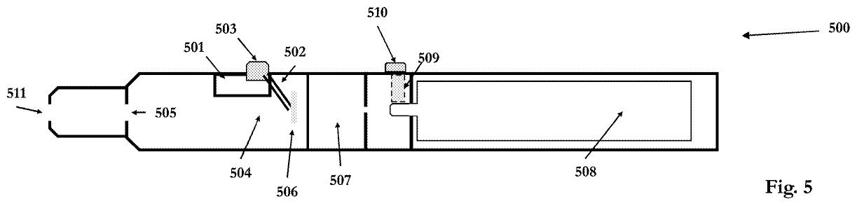

An exemplary embodiment (500) is shown in FIG. 5. It comprises a terpene container (501) configured to hold selected terpene composition, which container comprises at least one opening (502) and a terpene-release control (503), configured to release a selected amount of terpene-composition liquid and/or to release a selected amount of terpene-composition vapors from the terpene container (501) through said container opening (502). The exemplary apparatus further comprises a chamber (504), and said container is attached to said chamber so that said opening (502) is open to said chamber. In the exemplary apparatus the chamber (504) comprises at least one opening (505), which chamber opening is configured to provide terpene-enriched aerosol. The exemplary apparatus further comprises an adsorption unit (506), configured to adsorb terpene-composition liquid, wherein said terpene container (501) and said terpene-release control (503) are configured to release the terpene-composition liquid on to the adsorption unit (506). The exemplary apparatus further comprises a unit and/or container (507), configured for holding a therapeutic composition comprises at least one non-terpene volatile component.

The exemplary apparatus further comprises a spray unit (508) configured to generate aerosol comprises said terpene composition and said non-terpene volatile component, and a spray-release control (509), configured to release a selected amount of said aerosol; and wherein the spray-release control (509) and terpene-release control (503) are configured to operate in coordination. The exemplary apparatus further comprises a ratio-adjusting control (510), configured to adjust the ratio between the amount of terpene composition and non-terpene volatile component in the aerosol, wherein said ratio-adjusting control (510) and terpene-release control (503) are configured to operate in coordination. The exemplary apparatus further comprises an inhalation unit (511).

Thus, the scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An inhalation device, comprising:
(i) an inlet for vapors and/or for aerosol;
(ii) at least one terpene holding element configured to hold a selected terpene composition; and
(iii) an outlet for vapors and/or for aerosol
wherein said device is configured to receive vapors and/or aerosol through said inlet, move said received vapors and/or aerosol through said device, infuse terpene composition into said moved vapors and/or aerosol and generate and emit terpene-enriched vapors and/or aerosol through said outlet, wherein at least one of said inlet, said holding element and said outlet is configured to limit the concentration of terpenes in said emitted terpenes-enriched vapors and/or aerosol to less than one percent by weight, when said moved vapors and/or aerosol is at 10 degrees Celsius and at atmospheric pressure.

2. The inhalation device of claim 1, wherein at least one of said inlet, said holding element and said outlet is configured to adjust the concentration of terpenes in said emitted terpenes-enriched vapors and/or aerosol to at least one percent by weight, when said moved vapors and/or aerosol is at 30 degrees Celsius and at atmospheric pressure.

3-5. (canceled)

6. The inhalation device of claim 1, wherein said terpene holding element is a perforated material and wherein said held terpene composition is dispersed in said perforated material.

7-8. (canceled)

9. The inhalation device of claim 1, wherein at least a fraction of said moved vapors and/or aerosol passes through said terpene holding element.

10. The inhalation device of claim 1, wherein said terpene holding element comprises a container, wherein said container comprises at least one opening and wherein said terpene holding element comprises an element made of fibers, and wherein said element is at least partially connected to said opening.

11. (canceled)

12. The inhalation device of claim 1, wherein said terpene holding element comprises a container, wherein said container comprises at least one opening, wherein said container comprises a wall, wherein said terpene holding element comprises a moveable and flexible element in at least a part of said wall and wherein said flexible element is configured to release on movement a pre-determined amount of said terpene composition from said container.

13. (canceled)

14. The inhalation device of claim 10, wherein at least one of said at least one opening comprises a funnel element and wherein said funnel element is configured to generate suction, when moved vapors and/or aerosol pass through said inhalation device.

15. (canceled)

16. The inhalation device of claim 10, further comprising an activation module, wherein said activation module is a button or an electronic sensor, wherein said activation module is configured to release a pre-determined amount of said terpene composition from said container, when activated.

17-19. (canceled)

20. The inhalation device of claim 1, configured to attach to at least one of an enriched gas inhaler, a therapeutic inhalation device, an inhaler, a smoking device, a medical cannabis inhalation device, a cigarette and a cigar.

21-23. (canceled)

24. A method for treating a patient comprising providing an inhalation device according to claim 1, loading a selected terpene composition onto said holding element, generating said enriched vapors and/or aerosol and administering said enriched vapors and/or aerosol to said patient for inhalation.

25. (canceled)

26. An apparatus for enriching vapors and/or an aerosol with a selected amount of a selected terpene composition, comprising
(i) a terpene container configured to hold a selected terpene composition, which container comprises at least one container opening;
(ii) a terpene-release control, configured to release a selected amount of a terpene-composition liquid and/or to release a selected amount of terpene-composition vapors from said terpene container through said at least one container opening into vapors and/or aerosol in contact with said opening, generating thereby enriched vapors and/or an enriched aerosol, wherein at least one of said opening and said control is configured to limit the concentration of terpenes in said enriched vapors and/or an enriched aerosol to less than one percent by weight, when vapors and/or aerosol in contact with said opening is at 10 degrees Celsius and atmospheric pressure.

27. The apparatus of claim 26, wherein at least one of said opening and said control is configured to adjust the concentration of terpenes in said enriched vapors and/or an enriched aerosol to at least one percent by weight, when vapors and/or aerosol in contact with said opening is at 30 degrees Celsius and at atmospheric pressure.

28. The apparatus of claim 26, further comprising a chamber, wherein said container is attached to said chamber wherein said at least one container opening is open to said chamber, wherein said chamber comprises at least one chamber opening, which chamber opening is configured to provide said generated terpene-enriched vapors and/or terpene-enriched aerosol, and wherein said chamber optionally comprises an adsorption unit, configured to adsorb terpene-composition liquid, wherein said terpene container and said terpene-release control are configured to release said terpene-composition liquid on to said adsorption unit.

29. (canceled)

30. The apparatus of claim 28, further comprising a holding unit configured for holding a selected therapeutic composition comprising at least one non-terpene volatile component, wherein said therapeutic composition comprises at least one cannabinoid.

31-33. (canceled)

34. The apparatus of claim 28, further comprising
(i) a heating unit configured to heat said therapeutic composition;
(ii) a heating control
wherein said heating unit is configured to heat said therapeutic composition to a temperature sufficient for evaporating a selected amount of said non-terpene volatile component, whereby vapors of said volatile component are formed.

35-36. (canceled)

37. The apparatus of claim 34, configured so that the ratio between the selected amount of evaporated therapeutic composition and the selected amount of terpene-composition is adjustable, wherein said apparatus optionally comprises a ratio-adjusting control and wherein said ratio-adjusting control and said terpene-release control are configured to operate in coordination.

38-39. (canceled)

40. The apparatus of claim 34, wherein said heating unit is configured to heat said chamber to a temperature sufficient for maintaining in vapor form at least a fraction of said non-terpene volatile component.

41-50. (canceled)

51. The apparatus of claim 26, wherein said terpene-release control, is configured to release between 0.01 milligram and 100 milligram of terpene composition from said terpene container.

52-54. (canceled)

55. A method for treating a patient comprising providing an apparatus according to claim 34, wherein said terpene container comprises a selected terpene composition, wherein said therapeutic composition holding unit comprises a selected therapeutic composition comprising at least one non-terpene volatile component and operating said terpene-release control and said heating control, whereby terpene-enriched therapeutic composition vapors are provided.

56. A method for treating a patient comprising providing an according to claim 30, wherein said container comprises a selected terpene composition, wherein said holding unit comprises a selected therapeutic composition comprising at least one non-terpene volatile component and operating said terpene-release control and said spray-release control, whereby terpene-enriched therapeutic composition aerosol is provided.

57-98. (canceled)
